(19) United States
(12) Patent Application Publication
(10) Pub. No.: US 2011/0021922 A1
(43) Pub. Date: Jan. 27, 2011

(54) MONITORING THE FLOW OF FLUID

(75) Inventors: Nicolay Berard-Anderson, Oslo (NO); Gjermund Fjeld Olson, Oslo (NO)

Correspondence Address: SUTHERLAND ASBILL & BRENNAN LLP, 999 PEACHTREE STREET, N.E., ATLANTA, GA, 30309, US

(73) Assignee: NEORAD AS, Oslo (NO)

(21) Appl. No.: 12/680,237

(22) PCT Filed: Oct. 3, 2008
(86) PCT No.: PCT/GB08/03354

§ 371 Date: Sep. 23, 2010

(30) Foreign Application Priority Data
Oct. 3, 2007 (GB) 0719332.9
Oct. 11, 2007 (US) 60960738
Apr. 29, 2008 (GB) 0807861.0

(51) Int. Cl. A61B 8/06

(52) U.S. Cl. 600/454

(57) ABSTRACT

A method of monitoring the intended injection of fluid into a blood vessel during a scanning or imaging procedure, the method including the steps of receiving an indication of the start of an injection of fluid into the blood vessel, measuring the flow velocity in the blood vessel downstream of the point of injection, and automatically controlling the scanning or imaging procedure based on the measured flow velocity.

The invention relates to monitoring the flow of a fluid in a fluid conduit in the human or animal body.

There are many situations where a non-invasive measurement of fluid flow in fluid conduits in the human or animal body is desirable. Such fluid flows may include flow velocity in blood vessels (such as arteries and veins), urinary tracts, gastro-intestinal ducts (such as the bile duct) and so on. Measuring the fluid flow velocity gives knowledge of the physiological condition of a patient, and can help with identifying potential health problems. Measuring the flow velocity can also help in determining the effect of procedures that introduce fluids into conduits of the body, such as the injection of contrast medium.

Often, contrast medium is injected or infused in procedures such as angiography, computed tomography, ultrasound and MRI. It is necessary to inject the contrast medium at high flow rates for a long time period, and to do this it is injected under pressure rather than being simply gravity fed. Other procedures that require fluids to be injected in a similar manner include, for example, injections during the course of chemotherapy treatments. To aid in performing such injections a number of injector-actuated syringes and power injectors have been developed.

When fluid is injected into a blood vessel under pressure there is a risk of extravasation occurring. Extravasation is the accidental infusion of fluid such as contrast medium into tissue surrounding a blood vessel, rather than into the blood vessel itself. Extravasation can result from various situations. A common cause is operator error in placement of the needle, or movement of the needle caused by patient movement, which results in the needle effectively missing the blood vessel. In this case the injected fluid is injected into the surrounding tissue instead of into the blood vessel. Extravasation can also occur through the inability of the blood vessel to tolerate the rate of injection of the fluid resulting in leakage through the walls of the blood vessel into the surrounding tissue. This is especially the case with older patients who can have brittle veins, or patients undergoing chemotherapy treatment. If extravasation occurs then the injection and the associated procedure often has to be aborted and repeated at a later stage.

Clearly, it is beneficial to be able to detect extravasation in order to prevent pain or injury to the patient and to allow the injection procedure to be stopped if required. It is also beneficial to be able to monitor an injection to detect other problems that may occur during the course of the infusion of fluid, for example an equipment failure.

The applicant's earlier application, WO 2004/052431, discloses the use of an ultrasound Doppler technique to monitor an injection and provide a signal to indicate the possibility of extravasation. The method described is based on the fact that during the injection process the flow velocity of fluid in the blood vessel increases. Normally the flow velocity in a peripheral vein is very low, but during contrast injection using a power injector the flow velocity increases significantly. A direct monitoring of the increased venous flow velocity induced by the infusion can be performed. A lack of a velocity increase when injection commences indicates that extravasation could be occurring and thus gives the operator an early warning, prior to the point when extravasation would be detectable from a visual inspection or by palpation. The device disclosed can send an automated signal to the power injector to halt the infusion if no velocity increase is detected, thereby limiting the risk of tissue damage.

However, although the system just described provided a major advance in the art, there are limits on how effectively it can measure the flow velocity within the blood vessel under certain conditions. A signal indicative of the start of injection or of a change in the flow rate can usually be detected, but during steady injection of fluid under pressure the Doppler signal can sometimes be too weak to provide a useful measurement. Further, with the prior system it is not possible to effectively monitor an injection under some conditions involving elevated flow rates and/or elevated concentration (viscosity) of the injected fluid.

Viewed from a first aspect, the present invention provides a method of monitoring the intended injection of a fluid into a blood vessel, the method comprising: conveying the fluid to a point of injection, introducing a disturbance into the fluid flow prior to injection, and monitoring the fluid flow in the blood vessel downstream of the point of injection using a Doppler ultrasound sensor.

By introducing a disturbance into the fluid flow the strength of the Doppler signal reflected from the fluid in the blood vessel is increased due to an increase in the number of reflection planes in the disturbed fluid flow. Thus, the disturbance introduced in the fluid flow prior to injection results in a disturbed flow in the injected fluid and/or blood in the blood vessel. This enables an injection to be more easily monitored at all stages. Further advantages are also obtained, such as the ability to monitor injection of fluids at an increased range of flow rates and fluid viscosities. As the disturbance is introduced into the fluid to be injected before it enters the body, there is no need for additional procedures to be carried out directly on the blood vessel itself, instead the disturbance is simply added to the fluid as it is injected.

The invention is particularly advantageous when used to monitor the venous injection of contrast medium, as contrast medium creates various problems for the prior art Doppler monitoring as discussed below. However, monitoring of the injection of any fluid can be carried out using the method and apparatus of the invention. The invention is also particularly useful in the monitoring of an injection to identify the onset of extravasation, but may also identify other problems with the injection process, such as a blockage or kink in the tubing conveying fluid to the patient.

In preferred embodiments, the disturbance comprises turbulence, eddies and/or cavitations in the fluid flow. The disturbance is thus created by exposing the fluid to changes in dynamic parameters, including but not limited to, pressure, velocity and direction. The disturbance may advantageously be introduced just prior to a cannula or Venflon™ that conveys fluid into the blood vessel.

Preferably, the disturbance comprises cavitation generated by a pressure reduction in the fluid flow, in particular a pressure reduction obtained by use of the Venturi effect. The method may include passing the fluid into an area of increased cross-section to suddenly decrease the pressure and hence initiate cavitation. A constriction prior to the area of increased cross section may be used to increase the pressure prior to the decrease

A splitter or baffle may be used to divide and/or mix the flow of fluid. This is thought to promote increased turbulence. Where a change in cross-section is used, a splitter may be placed just after the increase in cross-section.

The monitoring preferably comprises measuring the fluid flow velocity in the blood vessel downstream of the injection site. A successful injection of fluid with result in changes to the fluid flow velocity corresponding to expected changes, typically consisting of an increase in flow velocity when injection commences, followed by a sustained elevated velocity, and finally a drop in velocity when the injection halts. If the flow velocity does not increase when the injection commences, then this indicates a problem with the injection process, such as the needle missing the vein, or an equipment failure. Similarly, if an unexpected drop in flow velocity occurs during the course of an injection, then this indicates that a problem has occurred, for example there may be an extravasation resulting from a fragile blood vessel wall, patient movement may have dislodged or shifted the needle, or some failure of the injector equipment may have occurred.

The monitoring process may include providing feed back to the operator and/or feed back to a power injector that supplies the fluid to be injected. This enables a quicker response to potential problems in the injection process, and also the use of an automated response from the power injector if required. The feed back may include an indication of the fluid flow velocity in the blood vessel, for example a visual or aural indication. The feed back may include providing an alarm that indicates an unexpected change in the fluid flow velocity in the blood vessel, and/or providing an alarm that indicates the absence of an expected change in the fluid flow velocity. For example, if the rate of injection is constant and the fluid flow rate drops then an alarm may be provided. Alternatively, if the fluid flow rate does not increase when the injection begins or the rate of injection increases, then an alarm may be provided. The method may include sending a signal to a power injector to automatically stop the injection when a problem is detected.

The method may also include measuring a parameter of the injected fluid prior to injection, and providing an indication of an abnormality in the parameter. For example, the injection pressure or flow rate in the power injector could be measured, and a higher or lower value than expected may be used to provide an indication of a problem with the injector. This arrangement allows the operator to assess if a lack of a velocity increase or a drop in flow velocity is due to a failure of the injector mechanism rather than an extravasation or other problem with passage of fluid into the patient. For example, an increased pressure in the power injector could indicate that a pinched or kinked tubing is preventing fluid from reaching the patient. This measurement could be correlated with the Doppler ultrasound measurement of the flow velocity in the blood vessel, which would indicate a reduced velocity compared to the expected velocity. This could trigger an event-specific alarm or notification to the operator. Further, if a sudden pressure decrease in the power injector is correlated with the signal from the ultrasound sensor, and they are in accordance, it would suggest that an extravasation is about to occur, which again could trigger an alarm.

Viewed from a second aspect, the invention provides an apparatus for monitoring the intended injection of a fluid into a blood vessel, the apparatus comprising: a source of disturbance for introducing a disturbance into the fluid flow prior to injection, and a Doppler ultrasound sensor for monitoring the fluid flow in the blood vessel downstream of the point of injection.

The source of disturbance introduces a disturbance in the fluid such that the number of ultrasound reflection planes in the fluid is increased, thereby improving the signal strength received by the ultrasound sensor from the fluid in the blood vessel. Thus, as discussed above, without direct intervention in the flow of blood, a disturbance introduced into the injected fluid is used to provide a disturbance in the fluid flow in the blood vessel.

In a preferred embodiment, the source of disturbance comprises a source of turbulence, eddies and/or cavitations in the fluid flow. The source of disturbance thus preferably exposes the fluid to changes in dynamic parameters, including but not limited to, pressure, velocity and direction.

The source of the disturbance may advantageously be located just prior to a cannula or venflon that conveys the fluid into the blood vessel. In one embodiment, the source of the disturbance is located in tubing that conveys fluid to the cannula. With this arrangement the apparatus can be set up in the same way as a conventional arrangement for contrast medium injections. Alternatively, the source of the disturbance may be in an insert located between the tubing and the cannula. The use of an insert means that specially adapted tubing is not required.

Preferably, the insert includes a flow path arranged to provide a drop in pressure, to thereby initiate cavitation at the appropriate fluid flow rates. For example, a change in cross-section that uses the Venturi effect may be present. The insert may include an area of increased cross-section to suddenly decrease the pressure and hence initiate cavitation. A constriction prior to the area of increased cross section may be used to increase the pressure prior to the decrease

A splitter or baffle may be provided to divide and/or mix the flow of fluid. This is thought to promote increased turbulence. Where a change in cross-section is used, a splitter may be placed just after the increase in cross-section.

In a preferred embodiment the Doppler ultrasound sensor is arranged to measure the fluid flow velocity in the blood vessel. As discussed above, the fluid flow velocity is expected to have a certain profile when a successful injection occurs, and monitoring the velocity therefore enables problems with the injection to be identified.

The monitoring apparatus preferably includes a control system for providing feed back to the operator and/or feed back to a power injector that is providing the fluid for injection. The feed back can be an indication of fluid flow velocity measurements that result from possible problems with the injection. The control system may be for handling feed back as discussed above, and is preferably arranged to carry out such feed back processes.

The apparatus may include and/or may be integrated with the tubing and/or connectors that convey fluid from the source of injected fluid, generally a power injector, to the patient.

The apparatus may also include a sensor for measuring a parameter of the fluid to be injected, i.e. the fluid prior to injection, and a control device arranged to provide an indication of an abnormality in the parameter. The parameter may for example be a pressure or flow rate as discussed above.

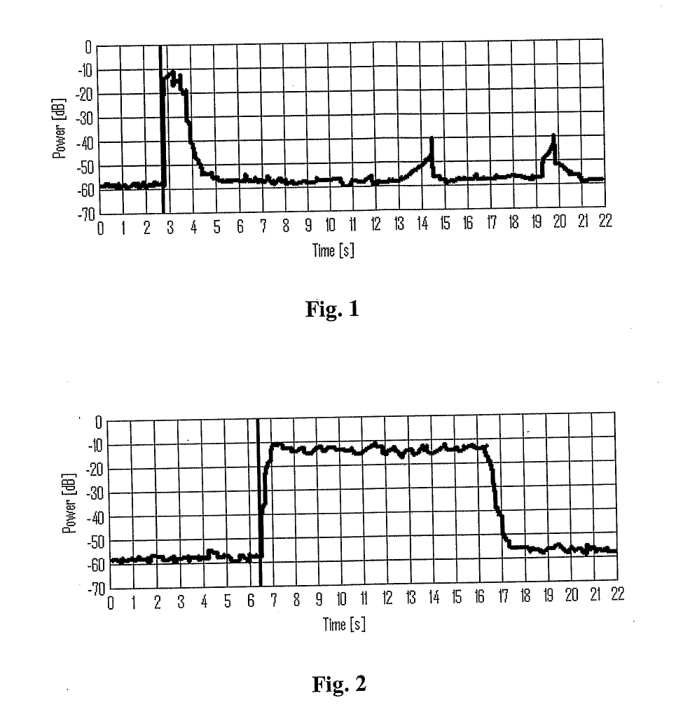

The method and apparatus of the invention allow continuous monitoring of the infusion induced venous flow, rather than only monitoring of certain stages of the injection process. The qualitative use of an ultrasound Doppler technique for monitoring of injection is enabled. Further, it is possible to monitor the injection of any concentration or viscosity of injected fluid, which is not possible using conventional Doppler injection monitoring as discussed below in relation to FIGS. 1 to 4.

As discussed above, the first and second aspects of the invention are based on the use of a source of disturbance in the fluid flow in order to increase the possibility of reflection of Doppler ultrasound waves. The use of a disturbance increases the signal strength for both low and high viscosity fluids. Doppler monitoring of injections at high concentrations and/or at higher flow rates, which was not previously possible, can be achieved effectively, and monitoring of the injection can occur at all stages of the injection process.

A further drawback with the prior art arises because the control of the injection process by the injection monitoring system described is based on quite basic feedback. In the prior art, if an increase in flow velocity is not detected when the injection of fluid begins, then either a notification is provided to alert the operator or a signal is provided which automatically shuts down the injector.

Viewed from a third aspect, the present invention provides a method of monitoring the intended injection of fluid into a blood vessel during a scanning or imaging procedure, the method comprising: receiving an indication of the start of an injection of fluid into the blood vessel, measuring the flow velocity in the blood vessel downstream of the point of injection; and automatically controlling the scanning or imaging procedure based on the measured flow velocity.

By automatically controlling the scanning or imaging procedure, unnecessary exposure of the patient to radiation can be avoided, and the scanning procedure may be carried out more effectively even if problems occur with the injection process. In addition, the amount of operator input is reduced, and thus the amount of work required to carry out an injection and scanning or imaging procedure is reduced

In a preferred embodiment, automatically controlling the scanning or imaging procedure comprises providing a first signal to stop the injection process if an expected rise in velocity does not occur within a set time; wherein the first signal also halts the scanning procedure.

Thus, if the monitoring device measures an increase in flow velocity during a set time from the start of the injection, it will allow the injection to proceed. This will ensure that the patient receives the prescribed contrast medium and the scanning process will be effective. However, if the expected increased velocity resulting from the injection is not obtained, it indicates that something is wrong and that the injection and also the scanning or imaging process should be terminated. The scanner may, for example, be an x-ray or MRI device. The inventors have identified that the need for the operator to manually shut down the scanning process, and the risk of the scanning process continuing despite the injection being stopped, presents a problem due to the extra work required by the operator and the risk of unnecessary exposure of the patient and operator to harmful radiation from the scanner. By halting the scanning procedure automatically when the injection is halted unnecessary radiation of the patient is avoided, and the attendant medical staff or the operator can approach the patient immediately without needing to manually control the scanner and without risk of exposure to radiation.

The method may comprise continuing to monitor the flow velocity if the expected rise in flow velocity occurs, and automatically controlling the scanning procedure by providing a second signal if there is a drop in the flow velocity, the effect of the second signal being dependent upon the size of the drop in flow velocity.

In a preferred embodiment, if the drop in flow velocity is larger than a preset limit. the effect of the second signal is to halt the scanning or imaging procedure. Thus, similar advantages to those discussed above are realised, as in the event of a possible extravasation or injector failure the patient and/or operator are not exposed to radiation unnecessarily and the scanner can be halted more quickly once a problem is detected. The preset limit may be as high as 100% of the signal strength, but preferably it is lower in order to allow the system to be triggered by partial failure in the injection process. For example, a drop of 50% or 25% of the steady state Doppler signal strength could be used to trigger the second signal.

Alternatively or in addition, the effect of the second signal may be to control a delay in the scanning or imaging procedure, the amount of the delay being dependent upon the size of the drop in flow velocity. Many scanning or imaging procedures operate after a certain time period from the beginning or end of the injection of contrast medium in order to allow time for the contrast medium to be transported to the desired imaging site by the body's circulation system. This could be time to allow the contrast medium to reach a particular organ or region of the body, or it could be the time required to allow the contrast medium to provide coverage of the whole body for a whole body scan. A useful image can result from the scanning procedure even if the contrast medium injection is not fully effected at the desired flow rate. Thus, if due to equipment failure or a small extravasation the contrast medium is not injected at the desired flow rate, this can be compensated for by adjusting the delay before the imaging process.

Further, in some cases, a timing bolus is administered/injected in order to estimate the patient's cardiac output to allow a calculation of the required time delay for a scanning procedure. In such an instance, the measurement of the flow velocity in the blood vessel can allow a calculation of cardiac output without the need for a timing bolus. This is true even if the flow velocity measurement is taking place on an extremity or peripheral part of the body. Once again, this will speed up the procedure and also avoid patient discomfort by reducing the number of injections required.

Thus, with this feature the time period before initiation of the scanning procedure may be controlled dependent on the flow velocity in the vein. The amount of delay will depend on the particular procedure. As an example, if a procedure was planned with a 20 second time period before commencing the scanning procedure, but the injection of contrast medium occurred at a rate 20% below the planned rate due to a partial extravasation or unexpectedly low cardiac output or some other problem, then the effect of the second signal might be a 5 second delay, leading to a 25 second time period before the scanning or imaging procedure begins.

Preferably, the first signal includes a signal that prompts the scanner to enter a recess or pause mode. This will enable the attendant medical personnel or operator to check the patient, the patient lines and the injector. Once any problems have been resolved the scanner will be able to restart immediately, making the procedure as efficient as possible, and avoiding the need for the operator to reset all of the systems.

In a preferred embodiment, the flow velocity in the blood vessel is measured using an ultrasound Doppler technique as described in WO 2004/052431, and discussed in more detail below.

The method may also include measuring a parameter of the injected fluid prior to injection, and providing an indication of an abnormality in the parameter as discussed above.

Viewed from a fourth aspect, the present invention provides an apparatus for monitoring the intended injection of a fluid into a blood vessel during a scanning or imaging procedure, the apparatus comprising: a flow velocity measuring device for measuring the flow velocity in the blood vessel downstream of the point of injection; and a control device that receives the measured flow velocity and receives an indication of the start of an injection of fluid into the blood vessel, wherein the control device is arranged to control the scanning or imaging procedure based on the measured flow velocity.

In a preferred embodiment, the control device is arranged to provide a first signal comprising a signal to stop the injection process if an expected rise in velocity does not occur within a set time, wherein the first signal also comprises a signal that halts the scanning or imaging procedure.

The scanner may for example be an x-ray or MRI device. The apparatus preferably includes connections to a control system of the scanner, such as wired connections or a wireless link. The content of the first signal may be adapted to suit different control mechanisms of the injector and scanner so that the first signal comprises the particular input signals required to control these devices. An interface may be used to convert or adapt the first signal to be a control signal for the injector and/or the scanner.

The control device may be arranged to continue to monitor the flow velocity if the expected rise in flow velocity occurs, and to control the scanning or imaging procedure by providing a second signal if there is a drop in the flow velocity, the effect of the second signal being dependent upon the size of the drop in flow velocity.

In a preferred embodiment, if the drop in flow velocity is larger than a set limit the effect of the second signal is to halt the scanning procedure. Alternatively or in addition, the effect of the second signal may be to trigger a delay in the scanning or imaging procedure, the amount of the delay being dependent upon the size of the drop in flow velocity.

Preferably, the first signal includes a signal that prompts the scanner to reset and enter a recess or pause mode.

In a preferred embodiment the flow velocity measuring device is an ultrasound Doppler device. The Doppler device may include one or more transducer elements.

The apparatus may also include a sensor for measuring a parameter of the fluid to be injected, i.e. the fluid prior to injection, with the control device being arranged to provide an indication of an abnormality in the parameter as discussed above.

In addition to the above problems with the prior art, there is a further issue that arises due to limitations in monitoring the further progress of an injection using the prior art system. Although continued monitoring of the injection after it has begun is contemplated in the prior art, no consideration is given as to how this should proceed. With the prior art, the injection of fluid and associated procedures such as angiography, computed tomography, ultrasound or MRI can therefore be halted unnecessarily or prolonged to the detriment of the patient.

Viewed from a fifth aspect, the present invention provides a method of monitoring the intended injection of fluid into a blood vessel, the method comprising: receiving an indication of the start of an injection of fluid into the blood vessel, measuring the flow velocity in the blood vessel downstream of the point of injection during the injection, and providing a signal if the measured flow velocity drops by more than a set limit, the effect of the signal being dependent upon the extent to which the injection of fluid has been completed.

By using a signal which is dependent upon the extent to which the injection of fluid has been completed the method can reduce the risks to the patient. In particular, if the injection process is close to completion when a problem occurs, it may be better to complete the injection along with any associated scanning procedure instead of stopping the procedure. This is because the risk of damage to the patient and the discomfort to the patient that would arise from repeating the procedure can be greater than the risk and discomfort involved in completing the procedure with an extravasation. For example, excessive exposure to contrast medium can damage the kidneys or other organs.

Thus, in a preferred embodiment the signal contains a signal to stop the injection of fluid if the measured flow velocity drops by more than a set limit prior to a set proportion of the injection being completed. This means that the risk of a prolonged extravasation or the continued exposure of the patient to radiation without effective measurements being taken by a scanner is avoided. Thus in this embodiment, the extent to which the injection has been completed is measured in terms of the proportion of the injection that has been completed.

If the measured flow velocity drops by more than a set limit after a set proportion of the injection is completed, then the signal may be a signal with no effect, but preferably it contains a signal that notifies the operator of the drop in the measured flow velocity. This means that the operator can assess the situation and either allow the injection and/or any associated scanning procedure to continue, or determine that the risk of continuing is the high or the benefit to the patient from continuing is too low and thus stop the injection manually.

For example, with this arrangement if the measured flow velocity drops after 25% of the injection has been completed, it is likely to be most beneficial to stop the injection and halt the scanner protocol, because proceeding with the remaining 75% with a possible extravasation could risk serious damage to the patient. However, if the measured flow velocity drops after 75% of the injection has been completed, it could be preferable to warn the operator instead of stopping the injection. In this case therefore the set proportion of the injection might be 50%.

The proportion of the injection may be measured in terms of the time of the injection, i.e. a proportion of the total duration of the injection, or it may be measured in terms of the volume of fluid to be injected. The method may include receiving an indication of the total duration of the injection, or of the total volume of fluid that needs to be injected. If the duration of injection is used, then the time since the start of the injection may be measured to thereby calculate the proportion of the injection that has been completed. If the volume of fluid is used, then the volume of fluid injected may be measured to calculate the proportion of the injection that has been completed. The flow rate of the injected fluid and the time since the start of injection may be used to calculate the injected volume and hence the proportion of the injection that has been completed.

The set proportion of the injection is preferably determined based upon the nature of the injection and of any associated scanning process. The method may include receiving an indication of the set proportion from a user or operator. The user preferably also inputs an indication of the total duration or volume of the injection.

The set limit for the drop in flow velocity may be a default value, or it may be input by the operator dependent upon the particular injection regime. The set limit could be as high as 100%, but preferably it is lower in order to allow the system to be triggered by partial failure in the injection process. For example, a drop of 50% or 25% of the steady state Doppler signal strength could be used to trigger the second signal. In the case of a 25% drop, if the steady state signal was 40 dB above the signal strength when measuring the vein with no injection, then the second signal would be triggered if the signal strength dropped by 10 dB.

Preferably, the method includes monitoring the flow velocity in the blood vessel downstream of the injection as the injection commences, and providing a first signal to stop the injection process if an expected rise in velocity does not occur within a set time; wherein if the injection process continues the method further comprises: continuing to measure the flow velocity in the blood vessel downstream of the point of injection and providing a second signal if the measured flow velocity drops by more than a set limit, the second signal being the signal based on the extent to which injection has been completed.

The method may also include measurement of a parameter of the fluid to be injected as discussed above. Thus, the operator can advantageously receive an indication of possible equipment failure. In the event that an injection is being allowed to proceed despite a possible extravasation or other problem, such an indication can allow the operator to more efficiently assess the equipment before deciding if the injection should be manually halted due to concerns about extravasation. For example, the operator might receive an indication of increased fluid pressure in the power injector, and could find that by checking and straightening the tubing conveying fluid to the patient that the dropped signal strength is recovered, allowing the procedure to continue.

Viewed from a sixth aspect, the present invention provides an apparatus for monitoring the intended injection of fluid into a blood vessel, the apparatus comprising: a flow velocity measuring device for measuring the flow velocity in the blood vessel downstream of the point of injection; and a control device that receives parameters including the measured flow velocity and an indication of the start of an injection of fluid into the blood vessel; wherein the control device is arranged to provide a signal if the measured flow velocity drops by more than a set limit during the course of the injection, the content of the signal being dependent upon the extent to which the injection of fluid has been completed.

The control device is preferably arranged such that the signal contains a signal to stop the injection of fluid if the measured flow velocity drops by more than a set limit prior to a set proportion of the injection being completed. If the measured flow velocity drops by more than a set limit after a set proportion of the injection being completed, then the signal may be a signal with no effect, but control device is preferably arranged such that it contains a signal that notifies the operator of the drop in the measured flow velocity.

The proportion of the injection completed may be based on a proportion of duration or of volume of the injection. Thus, the control device may be arranged to receive an indication of the total duration or volume of the injection and to determine the proportion of the injection completed using the time from the start of the injection and/or the flow rate of the injection as discussed above.

The set proportion of the injection is preferably determined based upon the nature of the injection and of any associated scanning process. The control device may be arranged to receive an indication of the set proportion from a user or operator.

Preferably, the control device is arranged to provide a first signal to stop the injection process if an expected rise in velocity does not occur within a set time after the start of the injection, and, if the injection continues, is arranged to provide a second signal if the measured flow velocity drops by more than a set limit, the second signal being the signal dependent upon the extent to which the injection of fluid has been completed.

There may be a sensor provided to measure a parameter of the fluid to be injected, with the control device being arranged to provide an indication when this sensor returns an abnormal reading as discussed above.

The method and apparatus of the third and fourth aspects can be advantageously combined with the method and apparatus of the fifth and sixth aspects. This provides injection monitoring that controls the operation of an associated scanning procedure to avoid unwanted exposure to radiation, and also ensures that an injection process and advantageously also an associated scanning procedure can be completed despite a potential problem with the injection if completing the procedure presents less of a risk to the patient.

It should be noted however that in some circumstances it may be beneficial to proceed with a scanning process even if the injection process is halted. This is because, depending on the particular procedure and patient condition, proceeding with the imaging process despite the possibility of a lower quality image due to an incomplete contrast medium injection can be better than stopping and restarting the procedure.

As will be appreciated, the monitoring of the third to sixth aspects will be most effective when a clear Doppler signal is present, especially in the case of an injection with a high flow rate and/or high concentration/viscosity. Thus, although the monitoring methods and apparatuses can be used with a conventional injection of fluid, the effectiveness of the system is restricted to a limited range of injection conditions. Therefore, in preferred embodiments the features of the first and second aspects and their preferred features are combined with the features of the third to sixth aspects. This provides an increased effectiveness and range of operation for the monitoring system.

The invention also encompasses computer program products containing instructions that when executed on a data processing apparatus will configure the data processing apparatus to carry out one of the methods discussed above. In a preferred embodiment this comprises software loadable onto or stored on a computer readable medium and consisting of computer readable program code for performing the method when the software is executed on a computer.

With any of the above aspects, monitoring the intended injection may include the storage of the measured blood vessel flow velocity data. For example, the data may be stored on an electronic storage medium such as a hard disk drive, or it may be output as a hard copy, such as a printout of velocity in a table or graph. Storing the data allows for a better record of the procedure, and the data can be accessed later for quality assurance purposes, or to improve the operation of the injection monitoring.

In preferred embodiments of the above aspects, an ultrasound probe having an array of two or more ultrasound sensors is used. The array is arranged to be placed substantially transverse to the direction of flow of the blood vessel so that at least one sensor of the array will be located over the vessel. With this arrangement, the accuracy with which the detector must be placed is reduced. It can be difficult to precisely locate the blood vessel downstream of the point of infusion. This arrangement makes it simple to obtain a good signal without time consuming repositioning of the probe. Provided that the ultrasound sensor is located approximately downstream of the infusion site, at least one sensor of the array will be located over the vessel, and so it is not necessary to precisely locate the blood vessel at the point of measurement prior to commencing the measurement. Instead, a change in flow velocity will be detected by whichever sensor or sensors of the array are located over the blood vessel.

With this arrangement, it is preferable that the sensor of the array that is located over the blood vessel is automatically detected using the difference in signal between sensors. The signal from an ultrasound sensors varies depending on whether the sensor is located over the blood vessel or over ordinary tissue. The highest signal will be obtained at the sensor in the array that is receiving measurements of the moving fluid in the blood vessel. This sensor can thus be selected to be used to monitor the intended injection.

In a particularly preferred embodiment, the signal received by the sensor of the array that has the lowest signal strength is used as a baseline signal level. This signal level is subtracted from the signal level of the sensor that is used to monitor the flow velocity in the blood vessel. The use of a baseline signal level in this way aids in noise cancellation, and will help reduce or eliminate erroneous signals caused by movement, palpation or other sources of noise, including external sources such as electrical devices. The baseline sensor may alternatively be a separate sensor intended to be positioned away from the blood vessel for the purpose of detecting signal changes due to movement and noise.

This latter feature is considered to be inventive in its own right even when not combined with an injection monitoring process for a blood vessel, and therefore, viewed from a further aspect, the present invention provides a method of monitoring flow velocity in a fluid conduit in the body using Doppler ultrasound measurement, comprising: locating a first ultrasound sensor over the conduit for monitoring the flow velocity, locating a second ultrasound sensor over body tissue, and using the signal from the second sensor to adjust the signal from the first sensor.

The signal of the second sensor may be used as a baseline signal to provide noise cancellation for the first sensor, which provides a signal for monitoring the flow velocity. Preferably, the signal level of the second sensor is simply deducted from the signal level of the first sensor.

The second sensor may be in a separate unit to the first sensor. However, preferably the first and second sensors are in an ultrasound probe formed of an array of two or more sensors. For example there could be three, four or five sensors, or more. The probe may be placed with the array located substantially transversely across the conduit, with one sensor that is over the conduit being used as the first sensor, and a sensor that is not over the conduit being used as the second sensor.

As discussed above, the first and second sensors may be selected from the array of sensors based on the signal level. The sensor in the array with the highest signal may be selected to be used to as the first sensor. The sensor in the array that has the lowest signal strength may be used as the second sensor. In a preferred embodiment, selection of the first and second sensors is carried out automatically by a processing unit, for example a CPU.

The method is of particular use in relation to blood vessels, and therefore the conduit is preferably a blood vessel.

Viewed from another aspect, the present invention provides an apparatus for monitoring flow velocity in a fluid conduit in the body using Doppler ultrasound measurement, comprising: a first ultrasound sensor for location over the conduit in order to monitor the flow velocity, a second ultrasound sensor for location over body tissue, and a signal processing device for using the signal from the second sensor to adjust the signal from the first sensor.

As with the method above, the processing device may be arranged to use the signal of the second sensor as a baseline signal to provide noise cancellation for the first sensor, which thus provides a more accurate signal for monitoring the flow velocity. Preferably, the signal level of the second sensor is simply deducted from the signal level of the first sensor.

The second sensor may be in a separate unit to the first sensor. However, preferably the apparatus, comprises an ultrasound probe formed of an array of two or more sensors, which includes the first and second sensors. The array could for example comprise three, four or five sensors, or more. The probe is preferably arranged to be placed with the array located substantially transversely across the conduit, with one sensor that is over the conduit being the first sensor, and a sensor that is not over the conduit being the second sensor.

The processing device may be arranged to select the first and second sensors from the array of sensors based on the signal level. The sensor in the array with the highest signal may be selected to be used to as the first sensor. The sensor in the array that has the lowest signal strength may be used as the second sensor.

The feature of detection of an abnormality in a parameter of the injected fluid, when combined with monitoring the flow velocity in a blood vessel during an intended injection, is also considered to be an invention in its own right. Thus, in still further aspects, the present invention provides a method or an apparatus for monitoring the intended injection of a fluid into a blood vessel, comprising monitoring the fluid flow in the blood vessel downstream of the point of injection, measuring a parameter of the injected fluid prior to injection, and providing an indication of an abnormality in the parameter. As noted above, correlation of an abnormality in the parameter and a change or a lack of change in flow velocity can be used to identify a problem with the injection process and to provide an indication of the source of the problem.

In all the embodiments discussed above, the ultrasound probe may include a microphone, and/or the ultrasound signal may be used as the basis for a sound signal. This enables audible feed-back to be provided. Where a microphone is used, the operator can simply listed to distinguish different types of blood flow, for example arterial and venous flow. When the ultrasound signal is used, this enables audible feed-back representing the change in ultrasound signal strength to be given to the operator. Consequently, the operator can listen for the expected change in strength as an injection commences, and/or monitor an ongoing injection by ear, with a change in signal strength resulting from a potential problem being audibly indicated, perhaps in addition to a visual indication.

Various aspects of the invention apply to monitoring an injection of fluid into a blood vessel. However, in some circumstances the fluid will never enter the blood vessel, and thus strictly speaking there is no injection of fluid into a blood vessel. For example if the needle is badly located and directly injects into tissue instead of into the blood vessel. Thus, in the aspects above, reference is made to monitoring an intended injection, in order to encompass monitoring of both an injection that is successful in introducing fluid into the blood vessel, as well as an injection that for whatever reason is not successful.

Preferred embodiments of the present invention will now be described by way of example only and with reference to the accompanying drawings in which:

FIG. 1 is a plot of Doppler signal strength when 100 mL of Optiray (320 mg I/mL) fluid is injected in a known way with a flow rate of 5 mL/s at room temperature,

FIG. 2 is a plot produced as for FIG. 1, but with 10 mL injected at an injection flow rate of 1.0 mL/s,

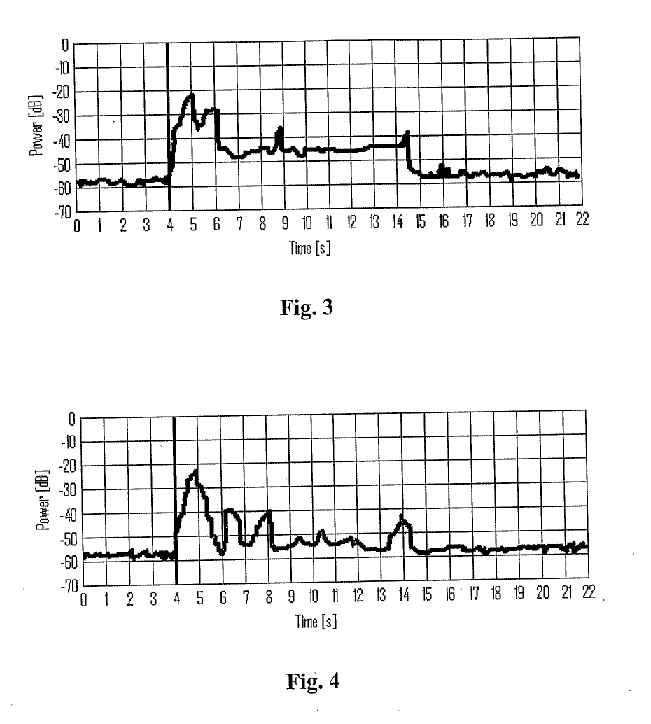

FIG. 3 is a plot produced as for FIG. 1, but with 30 mL injected at an injection flow rate of 3.0 mL/s,

FIG. 4 is a plot produced as for FIG. 1, but with 50 mL injected at an injection flow rate of 5.0 mL/s,

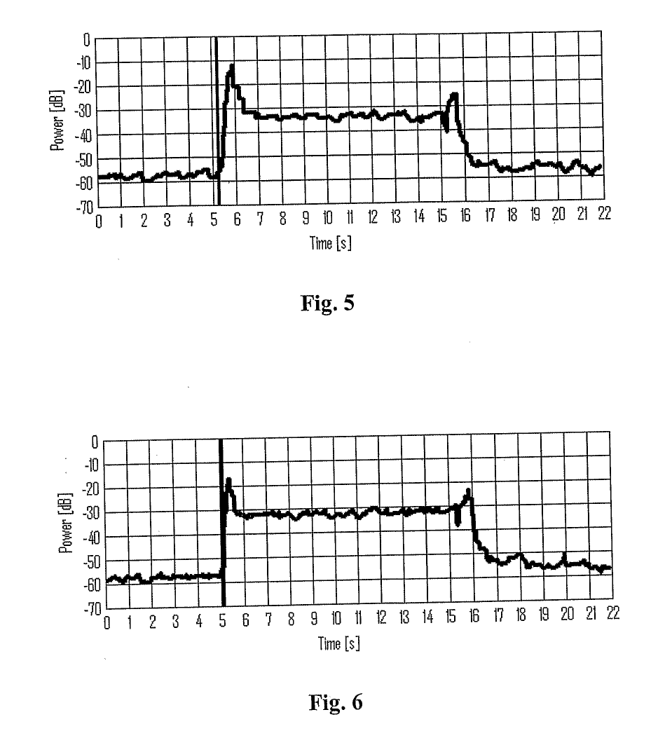

FIG. 5 is a plot of Doppler signal strength when 50 mL of Optiray (320 mg I/mL) fluid is injected with a source of disturbance in accordance with the invention, wherein the fluid has an injection flow rate of 5.0 mL/s,

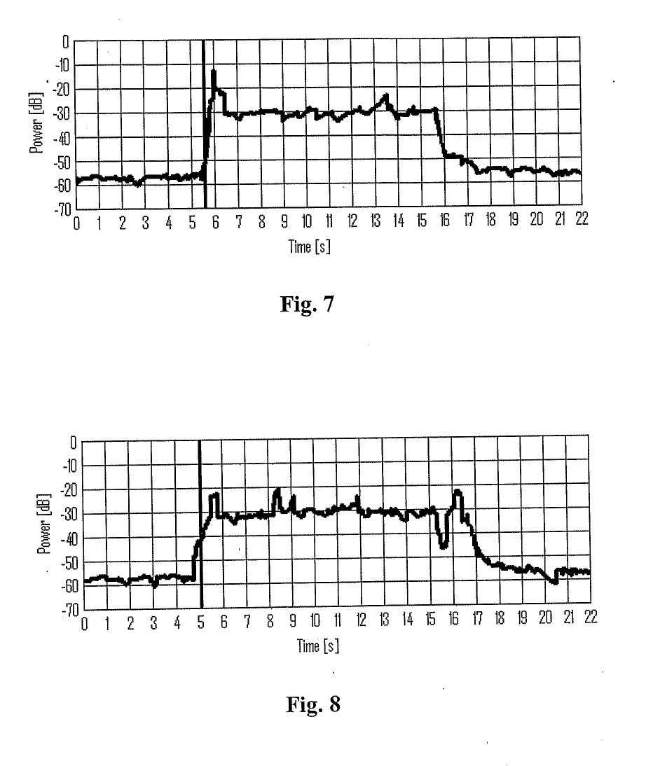

FIGS. 6 to 8 show the results of repetitions of the experiment of FIG. 5,

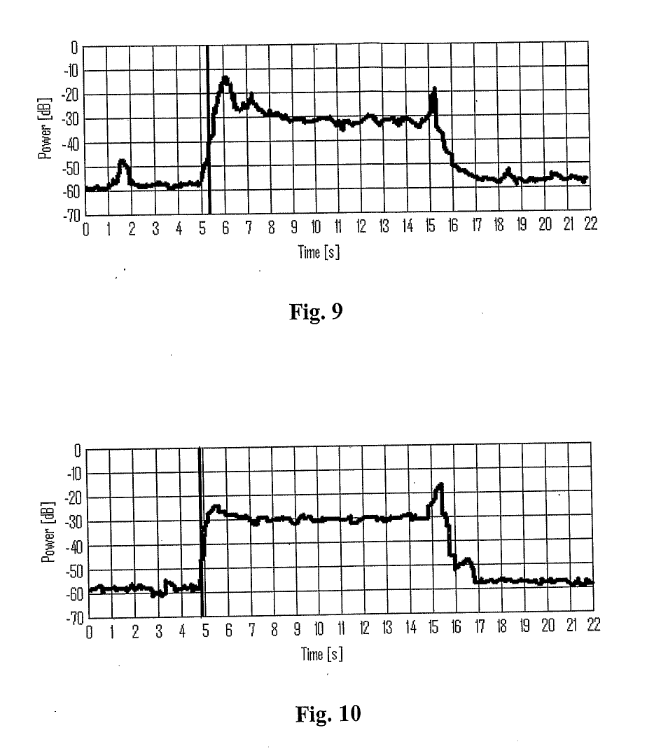

FIG. 9 is a plot of Doppler signal strength when 50 mL of fluid is injected as for FIG. 5, with the addition of a non-return valve in the simulated blood vessel, wherein the fluid has an injection flow rate of 5.0 mL/s,

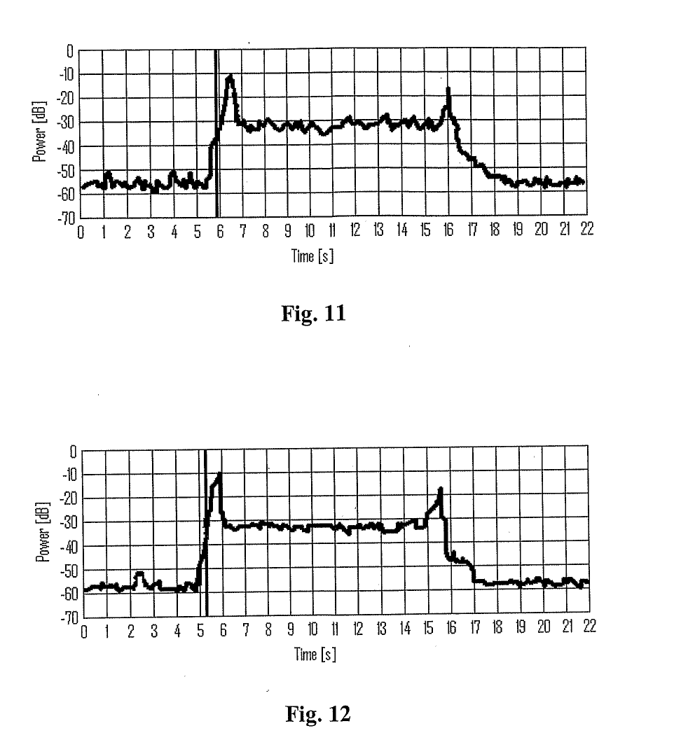

FIGS. 10 to 12 show the results of repetitions of the experiment of FIG. 9,

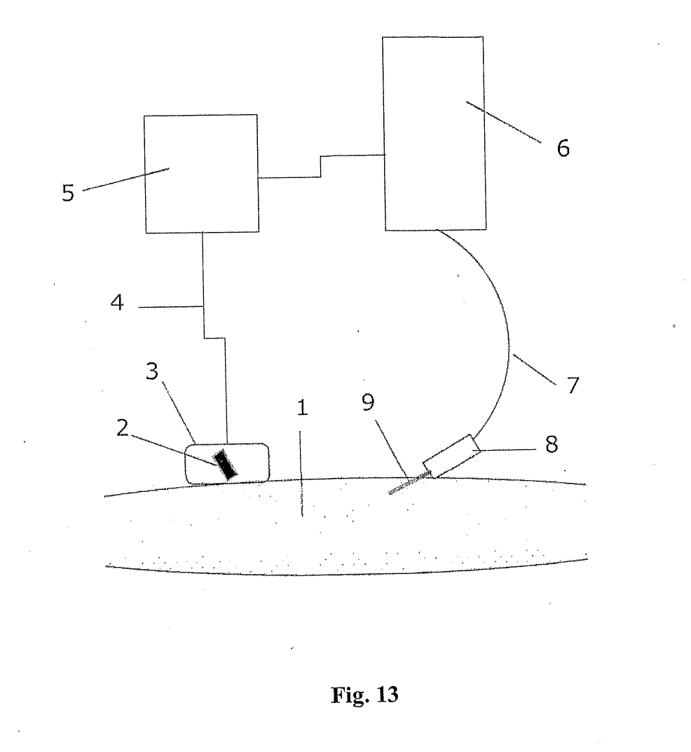

FIG. 13 shows schematically an arrangement for monitoring the injection of fluid in accordance with an embodiment of the invention,

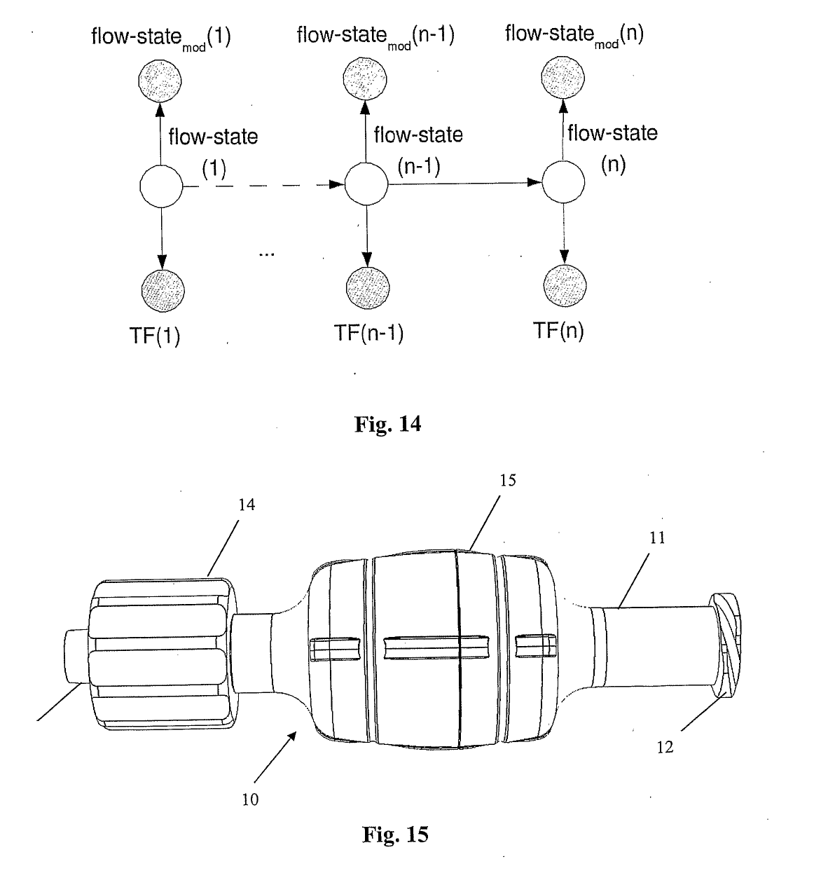

FIG. 14 shows a Bayesian network used in a system for processing and classification of the Doppler signal,

FIG. 15 illustrates an insert for introducing a disturbance in flow, and

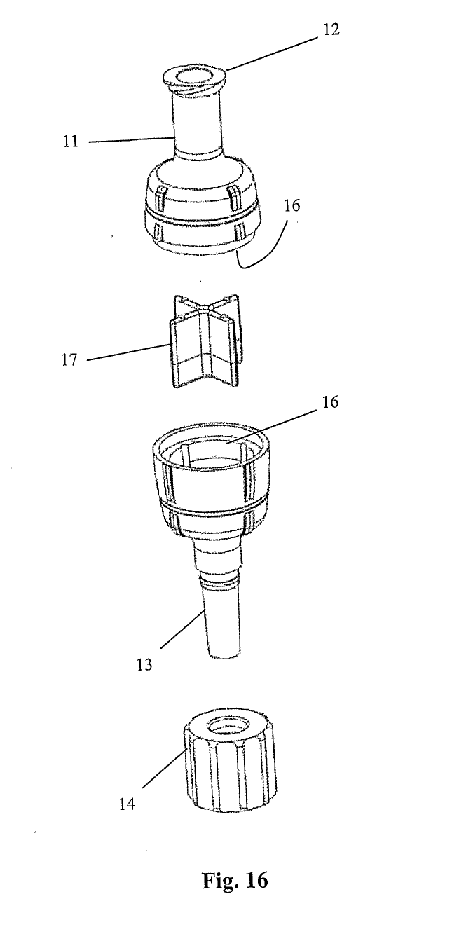

FIG. 16 is an exploded view of the insert of FIG. 15.

The inventors have found that the introduction of a disturbance, such as bubbles, eddies, cavitation and so on significantly increases the ability to measure fluid velocity by the use of Doppler ultrasound technology. This is particularly the case where the injected fluid would otherwise tend to form a homogeneous, laminar flow, which occurs, for example, with high flow rates of contrast medium.

In the Doppler ultrasound measurement of flow velocity an ultrasound Doppler probe is used, which consists of one or more transducer elements. The probe is located against the skin of a patient proximate to a vein into which the infusion is being made and downstream of the infusion site. The probe is fixed to the patient's skin using an adhesive. In accordance with standard practice, a coupling medium (ultrasound gel) should be applied to the patient's skin under the transducer elements. A signal indicative of the flow velocity in the blood vessel is produced from the Doppler probe in a conventional manner.

When monitoring contrast medium or other injections by an ultrasound Doppler method, the reflected signal strength depends on the degree to which the medium is able to reflect the ultrasound waves to the ultrasound detector. A Doppler transducer consists of one transmitter and one receiver. The transmitter emits a sound wave and a signal reflected from the measured substance is picked up by the receiver. An increase in the number of reflection planes in the flow of injected fluid can be induced by the addition of disturbance, which may be a disturbance in the form of turbulence, cavitations (micro bubbles), eddies or just plain mixing of different fluids. Thus, by introducing a disturbance into the injected fluid, the strength of the reflected Doppler signal is increased.

During contrast medium injection, or injection of other fluids, the blood flow velocity of the vein increases. Normally the flow velocity in a peripheral vein is very low, but during contrast injection using a power injector set at e.g. 5 ml/sec the flow velocity increases significantly. Using ultrasound Doppler technology this increased flow can be continuously monitored at the appropriate scale setting as long as the flow contains reflection planes to provide the required reflected signal. However, a regular injection of a homogeneous contrast medium into an inert medium creates very few reflection planes suitable for Doppler ultrasound. This is because the contrast medium will strongly displace any other liquid flow in the vein, leading to a flow of homogeneous contrast medium that does not readily result in mixing of the contrast medium with the blood. Very few reflection planes will be created without the presence of mixing with other fluids than contrast medium, and thus very poor reflected Doppler signals result, except at the start and end of injection, as discussed below in relation to FIG. 1.

Further, contrast medium is supplied by a number of manufacturers in a variety of concentrations. The osmolality and viscosity of the contrast medium varies with both concentration and temperature of the medium. The chemical structure of the medium will also influence the viscosity (such as non-ionic monomers or dimers in Iodine contrast medium). The surface chemistry will influence the behaviour of the medium when injected into a blood vessel. The diagnostic procedure and the patient physiology will determine the concentration and volume of contrast medium to be administered, as well as the flow rate. Certain required injection regimes cause problems for conventional Doppler monitoring as shown by FIGS. 1 to 4.

As illustrated in FIG. 1, a conventional injection of a homogeneous contrast medium into an inert medium using a high flow rate and high concentration creates very few reflections suitable for Doppler ultrasound. FIG. 1 shows readings collected using a ‘phantom’ set up to simulate injections into a blood vessel. The phantom consisted of a water filled box to simulate body tissue, and a silicone tubing to simulate the blood vessel, the tubing passing through the water filled box. The tubing used had a 3 mm internal diameter and a 5 mm external diameter. De-aerated water was passed through the silicone tubing to simulate blood. Contrast medium was injected into the tubing upstream of the phantom, and an ultrasound sensor of conventional type, such as the sensor discussed in WO 2004/052431, was placed to detect the flow velocity in the tubing within the phantom. The injected fluid was at room temperature and was introduced into the silicone tubing using a green venflon. The sensor was placed 4 cm from the venflon tip.

For FIG. 1, the injection flow rate was 5.0 mL/s, and 100 mL of Optiray (320 mg I/mL) was injected at room temperature.

The peaking signal strength at start and end of the injection is characteristic of injections of contrast medium through catheters. As a homogeneous fluid is injected through a venflon or catheter, it creates cavitations close to the tip for as long as the injection speed increases, and these cavitations provide the increased Doppler signal strength. However, as a steady state injection flow is established, very little cavitation occurs, and it is therefore not possible to effectively monitor the injection process. As the injection speed decreases at the end, the same phenomenon of cavitation occurs, leading to another peak.

During the start and end points, the flow can be characterised as turbulent. The turbulence creates reflection areas in the fluid, suited for measurement by Doppler ultrasound. However, between the start and end points of the injection shown in FIG. 1 the flow of infused contrast medium does not produce a Doppler signal. In this phase of the infusion the flow can be characterised as laminar. A homogeneous fluid in a laminar flow generates very little reflections. Thus, with this high concentration and high flow rate the conventional method can only monitor the injection process effectively at the start and end of the injection. Therefore, any problems occurring during the steady state phase cannot be detected.

FIGS. 2 to 4 show the results of a progressive increase in concentration and flow rate from a measurable low rate toward the high concentration and flow rate of FIG. 1.

Most concentrations at low injection flow rates result in a good mixing of blood and contrast medium, which induces a good Doppler signal. FIG. 2 shows the signal produced when an injection with a low flow rate of 1.0 mL/s and a low volume of 10 mL. As can be seen, an elevated signal strength is produced throughout the injection process showing that the conventional monitoring technique is capable of producing useful results with this injection regime.

However, higher flow rates, especially at higher concentrations (i.e. high viscosity), result in poor mixing with the blood. The flow tends to be more homogeneous, and it seems to displace the blood from the vein. There is also a great difference with respect to contrast medium temperature. A highly viscous contrast medium at room temperature tends to create a homogeneous/laminar flow with very few reflection planes compared to the same contrast medium at body temperature. Higher concentrations of viscous contrast medium will, because of the flow properties, form a plug which is much more difficult to push into the blood vessels. Some time after the injection starts, the vein will reach a maximum pressure limit for the injected flow rate, and the contrast medium will then start to flow backwards in the vein (from the venflon) as well as forwards. At this point, as there is no blood present in the vein, and hence no mixing to cause reflection planes, it is very difficult to obtain a good Doppler signal.

FIGS. 3 and 4 illustrate the deterioration in measured signal when the flow rate and volume are increased to 3.0 mL/s, 30 mL (FIG. 3) and then 5.0 mL/s, 50 mL (FIG. 4). It will be appreciated that in FIG. 4 the readings between the start and end phases of the injection are too low and erratic to provide a useful indicator of problems with the injection process.

As discussed above, to achieve an improved Doppler signal strength and allow monitoring of an injection throughout the course of the injection and also to allow monitoring of injections at increased flow rates and concentrations, a disturbance is introduced into the fluid flow prior to injection. Thus, a conventional arrangement for injecting fluid into a patient is used, with the exception that the cannula or venflon is provided with a source of a disturbance, such as a source of turbulence. To simulate this arrangement, experiments were performed as for FIGS. 1 to 4, but with the introduction of a source of disturbance in the venflon.

The effect of this modification to improve the Doppler signal strength can be seen from FIGS. 5 to 12. In the experiments shown the injection conditions of FIG. 4 were repeated, first with a non-return valve in the liquid flow prior to injection (FIGS. 5 to 8) and then with a further non-return valve in the liquid flow along the simulated blood vessel (FIGS. 9 to 12). The purpose of the second non-return valve is simply to ensure that the beneficial effects of the invention exist for varying conditions in the simulated blood vessel.

FIGS. 5 to 8 show that when a disturbance to the flow of the injected fluid is introduced, the signal strength is greatly improved compared to FIG. 4, which has an identical flow rate, volume and concentration. The non-return valve introduces turbulence as the fluid passes through it.

FIGS. 9 to 12 show that with a simulation of different blood flow conditions, using a non-return valve in the silicon tubing, the Doppler signal of sufficient strength is once again produced.

It will of course be appreciated that alternative devices to the non-return valve could be used to produce turbulence in the fluid prior to injection. For example an angle cock or a static mixer could be used. A preferred device is discussed in more detail below with reference to FIGS. 15 and 16. In addition, as discussed above, an alternative to a source of turbulence is a source of micro bubbles, which has the same effect of increasing the incidence of reflection planes to thus increase the Doppler signal strength.

Thus, as will be appreciated through the use of the phantom studies it has been shown that the introduction of a disturbance into the fluid flow prior to injection results in an improved Doppler signal, allowing monitoring of an injection throughout the entire course of the injection and also allowing injections and higher concentrations and higher flow rates to be effectively monitored. In the discussion above an obstruction placed in the liquid flow in front of the venflon, was used, and it was confirmed that this created enough turbulence to obtain a good Doppler signal. It shows a significant difference in the Doppler signal for high flow rate injections of contrast medium compared to the injection with no disturbance as shown in FIG. 4. The same phenomenon will occur when alternative turbulence sources are used or when micro bubbles are injected into the flow.

An embodiment of an injection monitoring system will now be described with reference to FIG. 13. A contrast medium is infused into the patient's arm 1 from a power injector 6. The power injector 6 is controlled by an electronic controller 5, which varies the injection speed as required and starts and stops the injector. The contrast medium flows via flexible tube 7 to a cannula or venflon arrangement 8, which comprises a connector for connection to the flexible tube, and a fine bore tube 9 which has been inserted into a vein in a known manner. A source of disturbance is in the tube 7 or the cannula arrangement 8 to introduce turbulence or the like. The source of disturbance could alternatively be in a separate interconnection unit, placed between and providing a fluid connection between the tube 7 and venflon 8. As in the above examples, the source of disturbance could be a non-return valve. In preferred embodiments the source of disturbance is an insert as described below with reference to FIGS. 15 and 16.

An ultrasound Doppler probe 3 is placed above the same vein at a convenient distance downstream so as to be clear of the infusion site. The Doppler probe 3 consists of a number transducer elements 2 which in use are placed at an angle to the vein to create and detect a Doppler shift resulting from the flow of fluid in the vein.

The transducer elements 2 comprise ultrasound sensors arranged in an array, which is placed substantially transversely over the vein. The signal levels of the sensor elements in the array is measured, and this is used to select appropriate sensors to measure the flow velocity and to provide a baseline signal for noise cancellation. The sensor in the array with the highest signal is selected to be used to as a first sensor, which measures the flow velocity. The sensor in the array that has the lowest signal strength is used as a second sensor, for noise cancellation. As will be appreciated, the sensor with the lowest signal level will be a sensor that is not affected by the movement of fluid in the blood vessel. The signal level of the second sensor will be the result of background reflections from body tissue and noise from external sources and from body movement. This baseline signal is deducted from the signal level of the first sensor in order to give a more accurate signal indicating the flow velocity in the blood vessel and changes in this flow velocity.

The probe 2 is connected via a flexible lead 4 to a controller 5 which includes a processor unit and display. This converts the output from the probe 3 into a form that may be displayed as an image on display unit in the conventional manner. In addition it provides a digital signal proportional to the flow velocity detected by the probe 3. This value is then also displayed on the display.

The controller 5 receives data concerning flow velocity and injection parameters, and provides a signal to the power injector 6 and a scanner as required in order to control the injection and an associated scanning procedure. It will be appreciated that various arrangements could be used to implement the required control device that monitors and controls the injection process. There can also be data passed back to the control device from the power injector, such as data from a pressure sensor, which can be used to indicate if there is a potential problem with the power injector or other injection.

The operator inputs the desired injection flow rate and the duration of the injection into the processor unit of the controller 5. The operator also inputs into the processor unit of the controller 5 the details of the proportion of the injection that triggers a change in the content of a second signal when a problem is detected during the course of the injection. An associated scanning process, such as angiography or MRI etc., is set up as required. When the injection is to commence the operator inputs a start signal into the controller 5. This in turn transmits a start signal to the controller 5 which energises the power injector 6 and causes it to run at the desired speed. The same start signal can also be used to co-ordinate initiation of the scanning process.

The processor unit then checks the flow velocity signal produced by the Doppler probe 3 as described above. If it is not satisfactory within a pre-determined short period of time, i.e. if the expected increase in velocity does not occur, then the infusion and associated scanning process will be stopped. This is achieved by sending a first signal to the controller 5 and also to the associated scanning equipment.

If the injection commences satisfactorily then the controller 5 continues to monitor the flow velocity signal. If an unexpected drop in flow velocity occurs then the controller 5 issues the second signal, the content of which depends on the proportion of the injection that has been completed and/or on the size of the drop in flow velocity. Thus, the processor unit also monitors the extent to which the injection has been completed, in this case the elapsed injection time as compared to the total duration of the injection.

In general, as discussed above, the second signal is a signal to halt the injection process as well as the scanning process if a flow velocity drop larger than a set limit occurs before the bulk of the injection has been completed, but if most of the injection has been completed then the second signal is a signal to produce a notification to the operator without stopping the injection process. As noted above, the set proportion of the injection at which the content of the second signal changes can be input by the operator before the injection is commenced, in order to allow the control of the injection to be tailored to the particular injection and scanning process. However, as an alternative or in addition to the possibility of customising this proportion, the processor unit may have a default proportion, such as 50%. Thus, in this situation, if a drop in flow velocity occurs prior to 50% of the injection being completed then the injection and scanning process is stopped, whereas if the drop in flow velocity occurs after 50% of the injection is complete, then the processor unit 4 issues a notification or alarm.

If the drop in flow velocity is smaller than the set limit for stopping the procedure, and the scanning procedure is one which utilises a waiting time prior to beginning, then the second signal can be used to delay the scanning procedure dependent upon the size of the drop in flow velocity as discussed above.

It will be appreciated that in general a drop in the flow velocity corresponds to a drop in the dB level of the signal provided by the Doppler sensor. Thus, in the foregoing discussion, references to the size of a drop in flow velocity correspond to measurement of the size in the drop in dB of the signal from the sensor.

The processor unit includes signal processing means to classify the Doppler measurements and identify anomalies. This can be done using known techniques, such as neural networks as discussed by Guler I. and Übeyli E. in “A recurrent neural network classifier for Doppler ultrasound blood flow signals”, Pattern Recognition Letters, Volume 27, Issue 13, 1 Oct. 2006, Pages 15601571 or Support Vector Machines (SVMs) as discussed by Übeyli E in “Doppler ultrasound signals analysis using multiclass support vector machines with error correcting output codes”, Expert Syst. Appl. 33(3): 725733 (2007). Bayesian classifiers have also been used to classify medical Doppler signals, as discussed in “Bayesian Classifier for Medical Data from Doppler Unit” by Malek J., Acta Polytecnica, Vol. 46, no. 4/2006. It is presently preferred however to use a pattern classification method combining a Bayesian network and a sparse kernel classifier, as described below.

In the system used with this method, the transducer elements 2 comprise three continuous wave Doppler 4 MHz transducers. These are attached to the patient's arm, over the vein and proximate to the contrast cannula. The transducer array is attached to the patient perpendicularly to the vein direction, circumferentially around the arm. A saline injection is administered first and then after a pause, the contrast agent is administered. Finally another saline injection is administered. There is a synchronization signal available that specifies whether the injection of fluid has started or ended and whether the injection is saline or a contrast agent.

First, pre-processing of the output signal from the transducer elements is carried out as follows. The transducer signal is quadrature demodulated, then sampled at 25 kHz and high pass filtered. The resulting power Doppler signal is short time Fourier transformed with a Hanning window of 0.2 seconds length and a two window length overlap. This results in a pre-processed 0.2 sec/2.5 Hz resolution time frequency signal. Finally, the 50 Hz band and its harmonics are removed.

After the pre-processing the inputs to the pattern classification system are:

1. three×2D pre-processed time frequency samples, and

2. a synchronization signal tuple {(start,stop), (contrast, saline)}

The classification system output is a state variable capturing if the injected fluid is running normally through a vein, and if nothing seems to flow or an extravasation may be occurring. The variable “flow-state” is hence defined as:

flow-state=(intravenous,noflow,extravasated)

The patient examination goes through several distinct phases. For the classifier, important phases are a possible pre-check phase where a saline injection is administered, a possible saline injection before the contrast injection, the contrast injection itself and finally a possible saline injection after the contrast injection. A variable “examination phase” is hence defined as:

examination phase=(saline-precheck,saline-preinjection,contrast-injection,saline-postinjection)

In a general dependency model it is assumed that the flow-state characteristically influences the received Doppler signal. A Temporal Feature (TF) is defined to be some representation of the Doppler signal at time t. The function TF(t) then statistically depends on the flow-state at time t.

It is also hypothesised that the chance for a particular flow-state is dependent on the flow-state at earlier times. For example, if it is not clear whether the flow is intravenous or extravasated during the saline-preinjection phase (e.g. the probabilities are roughly equal) then that might heighten the chance that the flow-state is seen as extravasated during the contrast-injection phase. Thus, the probability of a flow-state at time tₙ depends on the flow-state at time tₙ₋₁.

Finally, since the task is pattern classification, an independent flow-state probability estimate is defined, which stems from a classification model based on the Doppler signal. This variable, flow-state_{mod}, at time t depends on the flow state at time t. FIG. 14 shows these dependencies in terms of a Bayesian network. The circles are statistical variables. The arrows denote a statistical dependency. Shaded circles are observed variables and white circles are latent variables. The “ . . . ” and the arrow with dashed line on the left illustrate a chain of dependencies from time 1 to time n1.

Values of flow-state as it varies with time are defined as a vector flow-state(t), where flow-state(t)={flow-state (1), . . . , flow-state (n). Similarly, vectors flow-state_{mod}(t) and TF(t) are defined. The joint probability for the sequence of flow-states, model estimated flow-states and temporal features is then:

$$p ( flow \text{-} state , flow \text{-} {state}_{mod} , TF ) = {\sum }_{n = \text{?}}^{N} p ( TF ( n ) \vert flow \text{-} state ( n ) ) {\prod }_{n = \text{?}}^{N} p ( flow \text{-} {state}_{mod} ( n ) \vert flow \text{-} state ( n ) ) {\prod }_{n = \text{?}}^{N} p ( flow \text{-} state ( n ) \vert flow \text{-} state ( n - \text{?} ) ) \\ \text{?} \text{indicates text missing or illegible when filed} \qquad ( 1 )$$

The densities in equation (1) are assumed to be Gaussian, and so an expectation maximization algorithm can estimate the particular distributions in (1) given a set of observations, and a sum-product algorithm may be used to compute desired marginal distributions, as set out in “Pattern Recognition and Machine Learning” by Bishop C., Springer 2006, ISBN 0387310738.

A normal Bayes risk estimator can be used to make classification decisions, based on the marginal distribution p(flow-state(n)) and an appropriate loss function, which can be determined based on the particular procedure being undertaken. The Bayesian network thus captures the dependencies between latent variables, observed measurements, and previous system states.

The postprocessed Doppler signal is rich in frequency content, and it is thus reasonable to attempt to classify the flow-state based on a time-frequency representation of the signals. The Doppler baseband range is roughly 4 kHz. With the aforementioned 2.5 Hz resolution, there are some 1600 frequency coefficients per sample. The classification approach used should hence efficiently deal with high dimensional data in real time. Furthermore, it would be desirable if the classifier provides a confidence estimate, preferably as a probability that can directly serve as the p(flow-state_{mod}) estimate.

A sparse kernel machine such as a Support Vector Machine (SVM) is thus a possible classifier since such a machine capable of dealing efficiently with high dimensional data and is well suited for time series analysis. However, a preferred sparse kernel machine is a machine named the Relevance Vector Machine (RVM). This sparse kernel machine is based on a Bayesian framework and is described by Tipping, M. E., in “Sparse Bayesian learning and the relevance vector machine”, Journal of Machine Learning Research 1, 211-244. 2001. The RVM is used to classify the Doppler signals based on the Bayesian considerations set out above.

Briefly, the RVM estimates a conditional distribution:

p(flow-state|x,w,β)=N(flow-stately|y(x),β⁻¹) (2)

where x is the input vector, w is a parameter vector, N is the normal distribution and β is the noise precision. y(x) is defined in terms of a set of basis functions that with the RVM formulation is constrained to the Support Vector Machine (SVM)-like form:

$$y ( x ) = {\sum }_{n = \text{?}}^{N} {w}_{n} k ( x , {x}_{n} ) + b \\ \text{?} \text{indicates text missing or illegible when filed} \qquad ( 3 )$$

Here, in contrast to SVMs, the kernel k does not need to be positive-definite.

As set out in the paper by Tipping, on benchmark data the RVM has about equal accuracy as the SVM, training times are roughly an order higher, but there is no need to do exclicit parameter optimalization for RVM kernels. Classification of new data is significantly faster for RVMs than for SVMs.

The RVM is thus used to provide an effective probalistic estimate of flow-state using the Bayesian network set out above. This can then be used to set an output value of flow-state, and the control device can hence provide appropriate signals to the power injector and operator when a potential problem has occurred.

As noted above, FIG. 15 illustrates an example of an insert 10 for introducing a disturbance into the fluid flow prior to injection. The insert 10 is for placement prior to the catheter 8 shown in FIG. 13. The insert 10 has an inlet part 11 with a Luer thread 12 for fitment to the incoming fluid line 7, and an outlet part 13, fitted with a nut 14 so that it can be joined to an appropriate fitting to the catheter 8. The central part 15 of the insert 10, and the internal connections thereto, forms the source of disturbance for the fluid flow.

In FIG. 16, the insert 10 of FIG. 15 is shown in exploded view, and the internal arrangement of the central part 15 can be seen in more detail. A chamber 16 is formed between two halves of the body of the insert 10, and has a relatively large cross-sectional area. The inlet part 11 has a flow path with a relatively small cross-sectional area, which opens suddenly into the chamber 16. This hence forms an expansion chamber, with a pressure reduction resulting from the Venturi effect as fluid flows from the inlet part 11 into the suddenly increased cross-section of the flow path through the chamber 16, and this causes cavitation to occur when fluid is flowing at appropriate flow rates. To further disturb the flow, the chamber 16 is fitted with a splitter 17, which is a baffle with an X-shaped cross-section, that divides the flow into four parts, before it is recombined in the outlet part 13, and the fluid continues to the catheter 8.

By means of the insert 10 of FIGS. 15 and 16 a disturbance is introduced into the fluid flow in the form of cavitations and turbulence. As discussed above, the result of this disturbance is that when the injected fluid passes into the blood vessel, an increased number of reflection planes is present and the strength of the reflected Doppler signal is advantageously increased.

In some embodiments described above, an injection of contrast medium alone is described. However, as is known in the art, the contrast medium injection could occur in conjunction with an injection of saline, as described in connection with the preferred signal processing method. Such injections are achieved using ‘double-barrelled’ injectors. Saline can be used alone prior to or after the injection of contrast medium. If injected before the contrast medium the saline acts to open the vein, and prepare it for the contrast medium injection. When injected after the contrast medium the saline can be used to push the contrast medium further along the blood vessel by applying pressure behind the contrast medium. This is beneficial as it allows contrast medium at a higher concentration to be driven to a desired location for imaging, which might be some distance along the circulation system from the point of injection.

Further, as noted above, the invention, at least in some embodiments, is not limited to monitoring the injection of contrast medium, but also provides benefits when utilised during the intended injection of other fluids such as cytotoxins, anaesthetics, chemotherapy drugs and so on.

As will be appreciated, the flow detection apparatus of the invention is inherently capable of producing a measurement of a blood flow velocity, or other fluid flow velocities in fluid conduits in the human or animal body. Therefore, the processing unit 4 may usefully be provided with the ability to provide a straightforward velocity measurement, in addition to having the capability to detect extravasation and the like as discussed above.

The system shown in FIG. 13 is illustrated as using a wired connection 3 between the probe 2 and controller 5. In an alternative embodiment, data transmission between the probe 2 and controller 5 is by a wireless connection. The use of wireless data transmission is useful as it avoids a potential entanglement risk, and gives more freedom of movement by the patient and around the patient. Further, the various processing units and control devices of the embodiments described and claimed can be a single unit, such as a CPU, or could for convenience be separated. For example, local processing of ultrasound sensor signals could occur at the probe, enabling the first and second sensors to be selected and the baseline signal used to adjust for noise without the need to transmit data to and from the processing unit or control device that controls the power injector.

Another modification to the system of FIG. 13 involves the use of sound. As is well known, venous and arterial flows have distinguishable sounds. A microphone can be provided with or as part of the ultrasound probe in order to enable the sound of blood vessels at the location of the probe to be heard. These sounds can be provided to the operator by head phones or by a speaker system. This allows the operator to more easily position the probe over the desired blood vessel.

In addition, the variation in signal strength from the ultrasound probe during the course of a procedure can be relayed as sound to the operator, i.e. the ultrasound signal can be used as the basis for a sound signal. This allows any the change in signal strength resulting from correct operation or from potential problems to be presented as audible feed-back to the operator. The audible feed-back can be used in addition to a visual signal and/or a separate alarm.

We claim:

1. A method of monitoring the intended injection of fluid into a blood vessel during a scanning or imaging procedure, the method comprising: receiving an indication of the start of an injection of fluid into the blood vessel, measuring the flow velocity in the blood vessel downstream of the point of injection; and automatically controlling the scanning or imaging procedure based on the measured flow velocity;
wherein automatically controlling the scanning or imaging procedure comprises providing a first signal to stop the injection process if an expected rise in velocity does not occur within a set time; wherein the first signal also halts the scanning or imaging procedure; and
further comprising continuing to monitor the flow velocity if the expected rise in flow velocity occurs, and automatically controlling the scanning or imaging procedure by providing a second signal if there is a drop in the flow velocity, the effect of the second signal being dependent upon the size of the drop in flow velocity.

2. (canceled)

3. (canceled)

4. A method as claimed in claim 1, wherein if the drop in flow velocity is larger than a preset limit the effect of the second signal is to halt the scanning or imaging procedure.

5. A method as claimed in claim 1, wherein the effect of the second signal is to control a delay in the scanning or imaging procedure, the amount of the delay being dependent upon the size of the drop in flow velocity.

6. A method as claimed in claim 1, wherein the flow velocity in the blood vessel is measured using an ultrasound Doppler technique.

7. An apparatus for monitoring the intended injection of a fluid into a blood vessel during a scanning or imaging procedure, the apparatus comprising: a flow velocity measuring device for measuring the flow velocity in the blood vessel downstream of the point of injection; and a control device that receives the measured flow velocity and receives an indication of the start of an injection of fluid into the blood vessel, wherein the control device is arranged to control the scanning or imaging procedure based on the measured flow velocity;
wherein the control device is arranged to provide a first signal comprising a signal to stop the injection process if an expected rise in velocity does not occur within a set time, and wherein the first signal also comprises a signal that halts the scanning or imaging procedure; and
wherein the control device is arranged to continue to monitor the flow velocity if the expected rise in flow velocity occurs, and to control the scanning or imaging procedure by providing a second signal if there is a drop in the flow velocity, the effect of the second signal being dependent upon the size of the drop in flow velocity.

8. (canceled)

9. (canceled)

10. An apparatus as claimed in claim 7, wherein the control device is arranged to provide a second signal in accordance with the method of claim 4.

11. An apparatus as claimed in claim 7, wherein the flow velocity measuring device is an ultrasound Doppler device.

12. A method of monitoring the intended injection of fluid into a blood vessel, the method comprising: receiving an indication of the start of an injection of fluid into the blood vessel, measuring the flow velocity in the blood vessel downstream of the point of injection during the injection, and providing a signal if the measured flow velocity drops by more than a set limit, the effect of the signal being dependent upon the extent to which the injection of fluid has been completed.

13. A method as claimed in claim 12, wherein the signal contains a signal to stop the injection of fluid if the measured flow velocity drops by more than a set limit prior to a set proportion of the injection being completed.

14. A method as claimed in claim 13, wherein if the measured flow velocity drops by more than a set limit after a set proportion of the injection is completed, then the signal does not stop the injection of fluid.

15. A method as claimed in claim 12, wherein if the measured flow velocity drops by more than a set limit after a set proportion of the injection is completed then the signal notifies the operator of the drop in the measured flow velocity.

16. (canceled)

17. (canceled)

18. A method as claimed in claim 12, including monitoring the flow velocity in the blood vessel downstream of the injection as the injection commences, and providing a first signal to stop the injection process if an expected rise in velocity does not occur within a set time; wherein if the injection process continues the method further comprises: continuing to measure the flow velocity in the blood vessel downstream of the point of injection and providing a second signal if the measured flow velocity drops by more than a set limit, the second signal being the signal based on the extent to which injection has been completed.

19. An apparatus for monitoring the intended injection of fluid into a blood vessel, the apparatus comprising: a flow velocity measuring device for measuring the flow velocity in the blood vessel downstream of the point of injection; and a control device that receives parameters including the measured flow velocity and an indication of the start of an injection of fluid into the blood vessel; wherein the control device is arranged to provide a signal if the measured flow velocity drops by more than a set limit, the content of the signal being dependent upon the extent to which the injection of fluid has been completed.

20. An apparatus as claimed in claim 19, wherein the control device is arranged such that the signal contains a signal to stop the injection of fluid if the measured flow velocity drops by more than a set limit prior to a set proportion of the injection being completed.

21. An apparatus as claimed in claim 20, wherein the control device is arranged such that if the measured flow velocity drops by more than a set limit after a set proportion of the injection is completed, then the signal does not stop the injection of fluid.

22. An apparatus as claimed in claim 19, wherein the control device is arranged such that if the measured flow velocity drops by more than a set limit after a set proportion of the injection is completed then the signal notifies the operator of the drop in the measured flow velocity.

23. (canceled)

24. (canceled)

25. An apparatus as claimed in claim 19, wherein the control device is arranged to provide a first signal to stop the injection process if an expected rise in velocity does not occur within a set time after the start of the injection, and, if the injection continues, is arranged to provide a second signal if the measured flow velocity drops by more than a set limit, the second signal being the signal dependent upon the extent to which the injection of fluid has been completed.

26-47. (canceled)

48. A computer program product containing instructions that when executed on a data processing apparatus will configure the data processing apparatus to carry out the method of claim 1.

49. (canceled)

50. (canceled)

51. An apparatus as claimed in claim 7, wherein the control device is arranged to provide a second signal in accordance with the method of claim 5.

52. A computer program product containing instructions that when executed on a data processing apparatus will configure the data processing apparatus to carry out the method of claim 12.
